(19) United States
(12) Patent Application Publication
(10) Pub. No.: US 2011/0280916 A1
(43) Pub. Date: Nov. 17, 2011

(54) STABLE HYDROALCOHOLIC ORAL SPRAY FORMULATIONS AND METHODS

(76) Inventors: Frank E. Blondino, Easton, PA (US); Howard Malitz, Flemington, NJ (US)

(21) Appl. No.: 13/188,937

(22) Filed: Jul. 22, 2011

Related U.S. Application Data
(62) Division of application No. 11/737,260, filed on Apr. 19, 2007, now abandoned.
(60) Provisional application No. 60/792,942, filed on Apr. 19, 2006.

(51) Int. Cl. A61K 9/00; A61K 31/5415

(52) U.S. Cl. 424/400; 514/226.5

(57) ABSTRACT

Stable formulations of an active pharmaceutical agent suitable for oral spray administration for absorption by the oral mucosa and related methods of preparation and administration of active pharmaceutical agent formulations are provided. Preferred embodiments of the invention provide formulations comprising an active pharmaceutical agent, a solvent, a buffer, and a viscosity modifying agent, wherein when a unit dose volume of about 25 to 400 mcL of the oral spray composition is sprayed, the spray has a median particle size diameter of about 40 to about 100 microns.

This application claims priority to U.S. Provisional Patent Application No. 60/792,942, filed on Apr. 19, 2006, the disclosure of which is incorporated by reference herein in its entirety.

### FIELD OF THE INVENTION

The field of the this invention is hydroalcoholic oral spray pharmaceutical formulations, methods of manufacturing such formulations, and their use for obtaining fast blood levels of the active ingredient via absorption to the systemic circulatory system through the oral mucosa in human and non-human mammals.

### BACKGROUND OF THE INVENTION

There are several limitations associated with administration of pharmaceutically active compounds through the gastrointestinal tract. The harsh environment of the gastrointestinal tract may alter the pharmaceutically active ingredient and decrease the available dosage. Metabolism by the liver may also limit the available dosage. Furthermore, patients with gastrointestinal sensitivities may have undesired side effects resulting from the gastrointestinal route of delivery. Oral sprays may provide substantial benefits compared to other modes of drug administration including faster appearance of the pharmaceutically active ingredient in the blood, improved dosage reliability, improved safety profile, and increased bioavailability.

In order to be effectively absorbed by the oral mucosa, oral transmucosal spray formulations must produce spray patterns of a suitable ovality and particle size, and be delivered in a suitable unit dose volume to ensure maximal absorption and avoid unintended administration through the gastrointestinal tract by swallowing. What is needed are stable oral spray formulations in suitable unit dose volumes which produce spray particles for rapid delivery of the active ingredient via absorption to the systemic circulatory system through the oral mucosa.

### SUMMARY OF THE INVENTION

Preferred embodiments of the invention provide stable spray formulations of an active pharmaceutical agent producing spray particles or droplets having an ovality and median diameter effective for administration to the systemic circulatory system through the oral mucosa. Preferred embodiments of the invention provide oral spray compositions comprising an active pharmaceutical agent, a solvent, and a viscosity modifying agent, wherein when a unit dose volume of about 25 to 400 mcL of the pharmaceutical composition is sprayed, the spray has a median particle size diameter of about 40 to about 100 microns.

Particularly preferred embodiments of the invention provide formulations comprising meloxicam and pharmaceutically acceptable salts thereof suitable for oral administration, and related methods of preparation and administration of meloxicam formulations. The invention provides stable, hydroalcoholic oral spray formulations in a simple, elegant format for fast onset of the active ingredient via absorption to the systemic circulatory system through the oral mucosa. In one embodiment, meloxicam is formulated in a hydroalcoholic, oral, propellant-free spray formulation at a concentration of about 0.1 to 2% w/w, more preferably 0.25 to 1%, and most preferably about 0.47% w/w. A particularly preferred hydroalcoholic meloxicam formulation embodiment comprises meloxicam, boric acid, potassium chloride, polyvinyl alcohol, ethyl alcohol, sodium hydroxide, and purified water.

Another embodiment of the invention provides a pharmaceutical composition comprising about 0.1 to 2% w/w of meloxicam, about 1 to 10 mg/g polyvinyl alcohol, and 100-300 mg/g of ethyl alcohol. Further embodiments of the invention provide hydroalcoholic, oral spray compositions comprising meloxicam, an alcohol, and a buffer.

In yet another embodiment of the invention, a pharmaceutically effective amount of meloxicam is delivered to the systemic circulatory system of a mammal via actuation of a spray pump adapted for administration of the formulations to the oral mucosal surfaces. Further embodiments of the invention provide preservative-free hydroalcoholic meloxicam formulations and methods for their preparation.

Additional embodiments of the invention provide methods of treating inflammation by administering to an animal or human subject an oral spray composition according to the invention. Preferred embodiments administer a spray volume of about 25-400 mcL, preferably about 50-200 mcL, and more preferably about 100 mcL to the oral mucosa. In another embodiment the spray volume is about 50 mcL. The volume of spray preferably contains a dose of meloxicam in the range from about 0.025 mg to about 2 mg per kg per day, preferably about 0.05 mg to about 1 mg per kg per day, and more preferably about 0.1 to 0.2 mg per kg per day.

Additional features and advantages of the invention will be set forth in the description which follows and will be apparent from the description or may be learned by practice of the invention.

### DETAILED DESCRIPTION OF THE INVENTION

Reference will now be made in detail to the presently preferred embodiments of the invention, which, together with the following examples, serve to explain the principles of the invention. It is to be understood that the application of the teachings of the present invention to a specific problem or environment will be within the capabilities of one having ordinary skill in the art in light of the teachings contained herein. Illustrative embodiments of the products of the present invention and processes for their preparation and use appear in the following examples.

Preferred embodiments of the present invention provide hydroalcoholic oral spray compositions comprising an active pharmaceutical agent, a solvent, and a viscosity modifying agent, wherein when a unit dose volume of about 25 to 400 mcl of the oral spray composition is sprayed, the spray has a median particle size diameter of about 40 to about 100 microns.

Further preferred embodiments of the present invention provide stable, essentially preservative-free pharmaceutical compositions which are hydroalcoholic solutions comprising a therapeutically effective amount of meloxicam. The preferred compositions do not resort to use of a preservative, but instead achieve inhibition of microbial growth by including an alcohol, preferably at least about 10% ethanol, in the formulation.

Meloxicam is a non-steroidal anti-inflammatory drug of the oxicam class. Chemically, meloxicam is defined as 4-hydroxy-2-methyl-N-(5-Methyl-1,3-thiazol-2-yl)-2H-1,2-benzothiazine-3-carboxamide, 1,1-dioxide. Although preferred embodiments of the invention relate to meloxicam, additional or other active pharmaceutical agents can be used in the spray delivery vehicles and methods of the invention.

The formulations according to the invention may also contain additional active pharmaceutical agents, or use such active pharmaceutical agents in place of meloxicam, such as, for example, analgesics, non-steroidal anti-inflammatory drugs (NSAIDs), corticosteroids, hyaluronan, and opioids including salts thereof. Anti-inflammatory drugs such as alosetron, anakinra, beclomethasone, betamethasone, budesonide, celecoxib, clobetasol, cromolyn, desoximetasone, dexamethasone, epinastic, etanercept, etoricoxib, flunisolide, fluocinonide, fluticasone, formoterol, hydrocortisone, hydroxychloroquine, ibudilast, ketotifen, mesalamine, methotrexate, methylprednisolone, mometasone, montelukast, nedocromil, olsalazine, prednisone, ramatroban, rofecoxib, salbutamol, salmeterol, salsalate, terbutaline, triamcinolone, valdecoxib, zafirlukast, including salts and mixtures thereof can also be included as active pharmaceutical agents in formulations of the invention.

Celecoxib is a highly selective COX-2 inhibitor which is more selective for COX-2 inhibition over COX-1. Celecoxib can reduce inflammation and pain while minimizing gastrointestinal reactions. In one embodiment, formulations of the invention may contain celecoxib. In another embodiment, methods of co-administering meloxicam and celecoxib are provided for reducing inflammation and pain, for example, before, during, or after a medical or dental procedure.

In addition, the following therapeutic classes are also suitable for inclusion in the formulations of the invention or use in place of meloxicam in formulations of the invention. Illustrative examples include analgesics such as acetaminophen; NSAIDS such as aspirin, diclofenac, etodolac, ibuprofen, indomethacin, ketoprofen, nabumetone, naproxen, piroxicam, salsalate, and sulindac, including salts thereof; corticosteroids such as betamethasone, hydrocortisone, methylprednisolone, mometasone, and triamcinolone, including salts thereof; and opioids such as codeine, hydrocodone, hydromorphone, morphine, oxycodone, and tramadol, including salts thereof.

Other suitable active pharmaceutical agents suitable for inclusion in the formulations of the invention include, but are not limited to, ondansetron, sumatriptan, zolpidem, tizanidine, ropinerole, insulin, glucose, and nitroglycerine and the active ingredients disclosed in U.S. Pat. Nos. 5,869,082; 5,955,098; 6,391,282; 6,110,486; 6,676,931; 6,969,508; 6,977,070; and 6,998,110 and U.S. patent application Ser. No. 09/537,118, the disclosures of which are incorporated herein by reference in their entirety.

Under stability analyses, the storage stable compositions of the present invention show remarkable maintenance of the initial active agent concentration and reductions in impurities as compared to previous formulations. For example, after four months at 40° C. and 75% RH, meloxicam concentration increased from 94% to 101%. Without being limited to any particular theory, the increased concentration of meloxicam is believed to be due to the evaporation of alcohol. The level of impurity B was less than 0.1% through eight weeks, 0.1% at twelve weeks, and 0.4% at four months when stored at 40° C. and 75% RH. The stability of formulations in accordance with preferred embodiments of the invention are believed to be superior to the prior art and other tested formulations as discussed below and shown, for example, in Tables 1-4.

As used herein, “storage stable” means liquid pharmaceutical formulations in which the concentration of the active ingredient is substantially maintained during storage stability testing, and degradation products and/or impurities which are typically observed in storage stability testing of such formulations are absent or significantly reduced during storage stability testing. In one embodiment, storage stability is determined at a temperature range from about 5° C. to about 80° C., and more preferably from about 15° C. to about 30° C. In another embodiment, storage stability is determined at a relative humidity (“RH”) range from about 30% RH to about 90% RH, and more preferably from about 65% RH to about 75% RH. Preferred time intervals for measuring storage stability range from about 1 week to 2 years, more preferably from about 2 weeks to about 4 months, and most preferably at intervals of 2 weeks, 4 weeks, 8 weeks, 12 weeks, and 4 months.

Preferred formulations of the invention contain about 15% ethanol and about 0.5% of a viscosity modifying (or enhancing) agent such as polyvinyl alcohol. Without wishing to be bound by theory, it is believed that the inclusion of ethanol inhibits microbial growth in the formulation and leads to increased stability of the formulation. Other alcohols such as benzyl alcohol, chlorobutanol, glycerol, the parabens (for example, butylparaben, methylparaben), phenol, phenoxyethanol, phenylethyl alcohol, and propylene glycol, may be used in place of ethanol for this purpose. Thus, in accordance with one embodiment of the invention, it is not necessary to include an antimicrobial component or agent to ensure safe storage without the proliferation of pathogenic molds, yeasts, or bacteria. In another embodiment of the invention, various antimicrobials which are suitable for use in foods and other ingestible substances are known in the art and can be used in the present invention. Examples include the parabens (butylparaben, methylparaben, and propylparaben), propyl-p-hydroxybenzoates, sodium benzoate, and sorbic acid including salts thereof. A preferred antimicrobial agent is sodium benzoate.

Various buffers and buffer salts used to maintain pH also are suitable for use in the present invention. The formulations according to the invention will typically have a pH of about 7.0 to 12.0, more preferably a pH from about 7.5 to about 9.5, most preferably a pH of about 8.5. Examples of buffers include ammonium hydroxide, carbonate, citrate, glycine, maleate, and phosphate, including salts thereof.

Preferred embodiments of the invention are directed to buccal spray formulations for fast onset of the active ingredient via absorption to the systemic circulatory system through the oral mucosa. Therefore, preferred spray formulations of the invention maximize absorption to the systemic circulatory system and minimize or avoid absorption by other body systems (e.g., lungs, digestive system). The size of the spray particles contributes to whether the particle is absorbed into body systems other than the oral mucosa/circulatory system (e.g., lungs). For example, smaller sized particles are more likely to be inhaled. By “buccal” herein we mean of, or pertaining to, the mouth and oral cavity, including but not limited to the oral mucosal surfaces of the tongue, cheeks, gums and/or sublingual surfaces.

In one embodiment, the percentage of the particles (droplets) of the spray formulation (e.g., after actuation of a spray pump) having a diameter of less than ten microns is less than about 10%, more preferably less than about 5%. In another embodiment, the median diameter of the spray particles is from about 20 microns to about 150 microns, more preferably from about 40 microns to about 100 microns, and most preferably about 50 microns (e.g., Tables 4 and 5).

The ovality of the spray pattern indicates whether the spray is symmetrical. It is believed that the more symmetrical the oval shape of the pattern of spray particles, the more likely the particles will evenly cover the oral mucosa. In accordance with a preferred embodiment of the invention, the ovality ratio of the pattern is between about 0.5 and about 2.0, more preferably between about 0.75 about 1.5 (e.g., Tables 5 and 6). In one embodiment, increasing the viscosity of the formulation decreases the ovality of the spray pattern. In another embodiment, an increased concentration of polyvinyl alcohol or other viscosity modifying agent decreases the ovality of the spray pattern making the spray more symmetrical.

In preparing the formulations of the present invention, the active meloxicam component or other active pharmaceutical agent is preferably incorporated into a hydroalcoholic solution. Preferably, water, borate buffer, and ethanol are used as solvents in the formulations of the invention. However, it is recognized that other solvents may be used (e.g., ammonium hydroxide buffer, carbonate, citrate, glycine, maleate, and phosphate, including salts thereof) and alcohols (for example, benzyl alcohol, glycerin, glycofurol, polyethylene glycol, propylene glycol, and sucrose) which aid in maintaining the pH of the formulation and solubilizing the active agent. Similarly, the use of polyvinyl alcohol as a viscosity modifying agent is only one possible option. Other viscosity modifying agents include such as acacia, alginic acid, bentonite, carbomer, carboxymethylcellulose, carrageenan, cellulose, ceratonia, cetyl alcohol, chitosan, colloidal silicon dioxide, ethylcellulose, gelatin, guar gum, hydroxyethyl cellulose, hydroxyethylmethyl cellulose, hydroxypropyl cellulose, hypromellose, magnesium aluminum silicate, maltitol, maltodextrin, medium-chain triglycerides, methylcellulose, polydextrose, polyethylene oxide, povidone, propylene glycol alginate, sodium alginate, stearyl alcohol, sucrose, tragacanth, trehalose, wax, and xanthan gum including salts thereof. The concentrations of these viscosity modifying agents can be adjusted to provide desired flow properties of the product by various methods, including those disclosed in <911> Viscosity, United States Pharmacopeia-National Formulary (USP 29-NF 24) (2006), is hereby incorporated herein by reference in its entirety.

The formulations also may contain an optional propellant for delivery as an aerosol spray, or can be propellant-free and delivered by a metered valve spray pump. Suitable propellants include, but are not limited to, hydrocarbons (butane, propane, etc.), chlorofluorocarbons (CFC-11, CFC-12, etc.), hydrofluorocarbons (HFA-134a, HFA-227ea, etc.), and ethers (dimethylether, diethylether, etc.).

Optional sweetening, taste masking, or flavoring agents such as Neotame®, sorbitol, Splenda® (sucralose), sucrose, or Sunett® (acesulfamate K) also may be added if desired.

Various flavors or flavoring agents may be included to impart a pleasant taste. A pleasant taste is particularly important when the formulation is intended for administration to children or animals. Numerous flavors that are commonly used in pharmaceuticals, foods, candies, and beverages are also suitable for use in the present invention. Examples include beef, bubble gum, chicken, fish, fruit, licorice, peppermint, and other flavors. Sweeteners such as Neotame®, sorbitol, Splenda® (sucralose), sucrose, or Sunett® can also be used to adjust the flavor of the formulation.

The formulations of the present invention can be prepared by various methods. One embodiment of a manufacturing method is as follows. Preferably, meloxicam is dissolved in a strongly alkaline solution to achieve dissolution at the concentrations in the stock solution. In one embodiment, borate buffer is added prior to ethanol. Without being bound by theory, it is believed that the borate buffer protects against a decrease in apparent pH that is observed when ethanol is added. Without borate buffer, it is believed that the addition of ethanol reduces the apparent pH and the meloxicam may precipitate out of solution.

### Formula:

| Item# | | mg/g |
|---|---|---|
| 1 | Meloxicam, BP | 4.65 |
| 2 | Boric Acid, NF | 0.77 |
| 3 | Potassium Chloride, USP | 0.93 |
| 4 | Polyvinyl Alcohol, USP | 5.00 |
| 5 | Ethyl Alcohol, Dehydrated, USP | 150.00 |
| 6 | Sodium Hydroxide, NF/FCC | 1.08 |
| 7 | Purified Water, USP | 837.57 |

### Formulation Solution Concentrations

| Item# | | mg/g |
|---|---|---|
| 8 | 0.93% (w/w) Meloxicam Stock Solution | 500 |
| 9 | 5% (w/w) Polyvinyl Alcohol Solution | 100 |
| 10 | Ethyl Alcohol, Dehydrated, USP | 150 |
| 11 | 50 mM Alkaline Borate Buffer, pH 8.4 | 200 |
| 12 | Hydrochloric Acid (aq) | As needed for pH |
| | | to 8.5 +/− 2 |
| 13 | Boric Acid Buffer | QS final weight |

### Process

### Step

A Make 0.93% meloxicam Stock Solution:
(a) dissolve 0.93 g of meloxicam, BP in 94 g of purified water, USP and 4.95 g of 1M sodium hydroxide solution to a 100 g solution;
(b) completely dissolve meloxicam and determine if the pH is 11.5; if the pH is 11.5, QS to 100 g with purified water;
(c) if the pH is not within the range of 11.5+/−0.2, then add 1M sodium hydroxide to adjust the pH to 11.5+/−0.2, QS with purified water to 100 g,
(d) mix well.
   B Make 1 M Sodium Hydroxide as follows for 1000 mL:
(a) transfer approximately 500 mL of Purified Water, USP;
(b) accurately weigh 40 g of sodium hydroxide, NF ad transfer to the 1 L volumetric flask;
(c) allow solution to cool and QS to 1 L;
(d) mix well.
   C Make 50 mM Alkaline Borate Buffer (pH 8.4) as follows for 200 mL:
(a) dissolve 12.37 g of boric acid, NF and 14.91 g of potassium chloride, USP in a 1000 mL volumetric flask with 750 mL of purified water, and dilute with water to achieve a volume of 1000 mL (solution C1);
(b) make 0.2 M sodium hydroxide by accurately weighing 0.8 g of sodium hydroxide, NF and transferring the resulting solution to a 100 mL volumetric flask containing 50 mL of purified water, allow solution to cool and QS to 100 ml with purified water, and mix well (solution C2);
(c) combine 50 mL of solution C1 with 8.6 mL of solution C2 in a 200 mL volumetric flask and QS with purified water to 200 mL;
(d) mix well.
   D Make 5% Polyvinyl Alcohol as follows for 100 g:
(a) combine 5 g of polyvinyl alcohol, USP with 95 g of purified water, USP in an appropriate sized vessel;
(b) stir with heat in order to dissolve the polyvinyl alcohol;
(c) allow to cool before use;
(d) mix well.
   E Make 0.2M Hydrochloric Acid solution as follows for 25 mL:
(a) pipette 2 mL of 10% Hydrochloric Acid, NF into a 25 mL volumetric flask containing 15 mL of purified water, USP;
(b) dilute to volume with purified water, USP;
(d) mix well.
   F Make 0.47% Meloxicam with 15% Ethyl Alcohol as follows for 100 g:
(a) transfer 50 g of 0.93% Meloxicam Stock Solution to an appropriate vessel;
(b) add 10 g of 5% Polyvinyl Alcohol Solution and mix well;
(c) add 20 g of pH 8.4 Alkaline Borate Buffer and mix well;
(d) add 15 g of Ethyl Alcohol, Dehydrated and mix well;
(e) adjust the pH to 8.5±0.2 with 0.2M Hydrochloric Acid;
(f) dilute to weight with pH 8.4 Alkaline Borate Buffer;
(g) mix well.

The preferred presentation of the product is in pharmaceutically acceptable glass, PET, or HDPE bottles with a capacity of between 1 and 100 mL. To ensure long-term photostability, amber glass can be utilized but may not be required. Additionally, if PET or HDPE is chosen, the bottle may be opaque to ensure long-term photostability. The product is preferably dispensed using a metered pump device capable of delivering between 25 and 400 mcL. In one embodiment, the pump and actuator are modified such that a spray is dispensed horizontally to the bottle. This will allow easy dispensing to the mouth of the patient. The actuator may include an extension to allow for delivery to the buccal area of humans or animals.

The present invention also provides methods of treating various conditions in a subject (e.g., osteoarthritis, rheumatoid arthritis, inflammation, gout, and pain). The methods include administering to a subject in need of treatment a pharmaceutical composition according to the invention. In one embodiment, the subject is a human; in another embodiment the subject is a non-human mammal, preferably selected from the group of dogs, cats, horses, cattle, sheep, and swine. The preferred storage stable meloxicam compositions can be administered to a patient in any suitable dosage range, for example, 0.025 mg to about 2 mg per kg per day, preferably about 0.05 mg to about 1 mg per kg per day, and more preferably 0.1 to 0.2 mg per kg per day.

It is to be understood that application of the teachings of the present invention to a specific problem or environment will be within the capability of one having ordinary skill in the art in light of the teachings contained herein. The present invention is more fully illustrated by the following non-limiting examples.

### Example 1

Four formulations were prepared for stability testing. Formula 1 is 0.47% Meloxicam with 0.5% Glycofurol (Table 1), Formula 2 is 0.47% Meloxicam with 0.5% PVA and 7.5% EtOH (Table 2), Formula 3 is 0.47% Meloxicam with 0.5% PVA and 15% EtOH (Table 3), and Formula 4 is 0.47% Meloxicam with 0.5% PVA and 15% EtOH in Borate Buffer (Table 4). The formulas were stored at 25° C./60% RH, 30° C./65% RH, 40° C./75% RH, and 5° C. The formulations stored at 25° C./60% RH and 40° C./75% RH were tested at the following intervals: 2 weeks, 4 weeks, 8 weeks (2 months), 12 weeks, and 4 months.

In addition to these conditions, a cycling study was performed where all three formulations were kept at 5° C. each night from approximately 4 pm to 8 am the next morning. The formulations were stored at 40° C./75% RH for the duration of the working day, approximately 8 hours. The formulations were not cycled on the weekends or holidays. The stability of the formulations was tested at 4 months after initiation of the cycling study.

The initial Meloxicam concentration in Formula 1 was 96.8% and dropped to 82.2% at the 4 month 40° C./75% RH time points. In addition, an Impurity B was identified from analytical testing. The impurity appeared at a concentration 0.1% at 2 weeks and increased to 0.7% at 4 months.

The initial Meloxicam concentration in Formula 2 was 95.0%. The concentration increased to 96.4% after 4 months at 40° C./75% RH. At 40° C./75% RH, the concentration of Impurity B was less than 0.1% through 8 weeks. The concentration of Impurity B was 0.2% at 12 weeks and 0.5% at 4 months. The level of Impurity B was lower in Formula 2 then in Formula 1.

The initial Meloxicam concentration was 94.0% in Formula 3 and increased to 100.8% after 4 months at 40° C./75% RH. The increase in concentration may be due to the concentrating of the formulation by evaporation of alcohol. At 40° C./75% RH, Impurity B was less than 0.1% through 8 weeks. The concentration of Impurity B was 0.1% at 12 weeks and 0.4% at 4 months. The concentration of Impurity B is Formula 3 was lower than that observed for Formulas 1 and 2.

The initial Meloxicam concentration was 100.5% in Formula 4 and remained unchanged after 2 months at 25° C./65% RH and 40° C./75% RH. At 40° C./75% RH, Impurity A was less than 0.05% through 2 months. The concentration of Impurity B is Formula 4 was lower than that observed for Formulas 1, 2, and 3. Overall degradation in Formula 4 was similar to Formula 3.

Two formulas were prepared for a spray study (Tables 5-6) both containing 0.47% Meloxicam and 15% EtOH. Formula 5 has a concentration of 0.5% PVA and Formula 6 has a concentration of 0.25% PVA. The following four characteristics of the formulas were observed: (1) percentage of particles that have a diameter less than 10 μm, (2) cumulative distribution Dv(50), (3) cumulative distribution Dv(90), and (4) ovality. The percentage of the diameter of particles less than 10 μm may indicate the percentage of the particles at risk of being inhaled into the lung. The higher the Dv value, the fewer particles that can be inhaled. For the formulations submitted, the amount of particles less than 10 μm is 1.6% for the Formula 5 and 2.4% for Formula 6.

The Dv (50) and Dv (90) values are size values corresponding to the cumulative distribution of particles at 50% and 90%. Thus, the Dv (90) represents a size below which 90% of the cumulative distribution occurs. From this it can be inferred that the Dv (50) value corresponds to the median diameter. The Dv (50) and Dv (90) for both formulas are very similar.

The ovality is defined as the ratio of Dₘₐₓ and Dₘᵢₙ. Dₘₐₓ is defined as the largest chord, in mm, that can be drawn within the spray pattern that crosses the COMw (i.e., center of mass of the spray pattern) in base units. Dₘᵢₙ is described as the smallest chord, in mm, that can be drawn within the spray pattern that crosses the COMw in base units. COMw is defined as the center of mass of the detected spray pattern, where each pixel's intensity is taken into account. With regard to Formulas 5 and 6, the closer the ovality is to 1.0, the more symmetrical the shape of the spray. Formulation 5 has an ovality of 1.26 and Formula 6 has an ovality of 1.51. The 0.5% PVA formulation has a more preferred ovality.

TABLE 1
Stability of Meloxicam 0.47% with Glycofurol

| Stability | Spray | Spray | SC/SW | % Label | Impurity | Other |
|---|---|---|---|---|---|---|
| Condition | Weight | Content | Ratio | Claim | B & C | Impurity |
| Initial | N/A | Bulk 0.484% | N/A | 96.80% | N/D | Unknown |
| | | RSD: 0.2% | | | | 0.7% |
| 25/60/2 wk, | 98.4 mg | 0.461 mg, | 0.00468 | 92.10% | <0.10% | N/D |
| n = 3 | RSD: 1.5% | RSD: 1.1% | RSD: 0.4% | | | |
| 25/60/4 wk, | 100.2 mg | 0.475 mg | 0.00474 | 95.00% | Imp. B: 0.18% | N/D |
| n = 3 | RSD: 2.5% | RSD: 2.3% | RSD: 0.5% | | | |
| 25/60/8 wk, | 91.6 mg | 0.427 mg | 0.00466 | 85.40% | Imp. B: 0.14% | N/D |
| n = 3 | RSD: 9.4% | RSD: 9.0% | RSD: 0.5% | | | |
| 25/60/12 wk, | 99.8 mg | 0.467 mg | 0.00468 | 93.50% | Imp. B: 0.20% | N/D |
| n = 3 | RSD: 1.0% | RSD: 1.3% | RSD: 1.0% | | | |
| 25/60/4 mo, | 100.7 mg | 0.473 mg | 0.00469 | 94.50% | Imp. B: 0.27% | N/D |
| n = 3 | RSD: 0.4% | RSD: 1.1% | RSD: 0.9% | | | |
| 25/60/4 mo, cycle | 90.5 mg | 0.418 mg | 0.00462 | 83.60% | Imp. B: 0.25% | N/D |
| n = 3 | RSD: 15.1% | RSD: 14.9% | RSD: 0.3% | | | |
| 40/75/2 wk, | 96.1 mg | 0.450 mg | 0.00468 | 89.90% | Imp. B: 0.10% | N/D |
| n = 3 | RSD: 2.5% | RSD: 2.4% | RSD: 0.3% | | | |
| 40/75/4 wk, | 86.0 mg | 0.411 mg | 0.00478 | 82.10% | Imp. B: 0.29% | N/D |
| n = 3 | RSD: 12.8% | RSD: 13.0% | RSD: 0.3% | | | |
| 40/75/8 wk, | 84.9 mg | 0.396 mg | 0.00466 | 79.20% | Imp. B: 0.33% | N/D |
| n = 3 | RSD: 10.1% | RSD: 9.8% | RSD: 0.4% | | | |
| 40/75/12 wk, | 86.0 mg | 0.402 mg | 0.00467 | 80.40% | Imp. B: 0.49% | N/D |
| n = 3 | RSD: 9.5% | RSD: 9.4% | RSD: 0.4% | | | |
| 40/75/4 mo, | 85.4 mg | 0.411 mg | 0.00481 | 82.20% | Imp. B: 0.67% | N/D |
| n = 3 | RSD: 8.0% | RSD: 8.1% | RSD: 2.0% | | | |

TABLE 2
Stability of Meloxicam 0.47% with 0.5% PVA and 7.5% EtOH

| Stability | Spray | Spray | SC/SW | % Label | Impurity | Other |
|---|---|---|---|---|---|---|
| Condition | Weight | Content | Ratio | Claim | B & C | Impurity |
| Initial | N/A | Bulk 0.475% | N/A | 95.00% | N/D | Unknown |
| | | RSD: 0.4% | | | | 0.5% |
| 25/60/2 wk, | 98.4 mg | 0.467 mg | 0.00475 | 93.40% | <0.10% | N/D |
| n = 3 | RSD: 0.5% | RSD: 0.7% | RSD: 0.3% | | | |
| 25/60/4 wk, | 98.6 mg | 0.481 mg | 0.00488 | 96.20% | <0.10% | N/D |
| n = 3 | RSD: 1.5% | RSD: 2.0% | RSD: 0.5% | | | |
| 25/60/8 wk, | 90.3 mg | 0.436 mg | 0.00483 | 87.20% | <0.10% | N/D |
| n = 3 | RSD: 11.5% | RSD: 11.9% | RSD: 0.5% | | | |
| 25/60/12 wk, | 90.5 mg | 0.430 mg | 0.00475 | 85.90% | <0.10% | N/D |
| n = 3 | RSD: 11.3% | RSD: 11.5% | RSD: 0.4% | | | |
| 25/60/4 mo, | 96.7 mg | 0.463 mg | 0.00479 | 92.70% | <0.10% | N/D |
| n = 3 | RSD: 2.4% | RSD: 3.6% | RSD: 1.8% | | | |
| 25/60/4 mo, cycle | 98.7 mg | 0.466 mg | 0.00472 | 93.10% | <0.10% | N/D |
| n = 3 | RSD: 5.4% | RSD: 5.0% | RSD: 0.4% | | | |
| 40/75/2 wk, | 99.1 mg | 0.476 mg | 0.00480 | 95.30% | <0.10% | N/D |
| n = 3 | RSD: 0.6% | RSD: 0.3% | RSD: 0.4% | | | |
| 40/75/4 wk, | 92.8 mg | 0.456 mg | 0.00491 | 91.20% | <0.10% | N/D |
| n = 3 | RSD: 11.9% | RSD: 12.0% | RSD: 0.9% | | | |
| 40/75/8 wk, | 96.6 mg | 0.462 mg | 0.00478 | 92.50% | <0.10% | N/D |
| n = 3 | RSD: 2.7% | RSD: 2.3% | RSD: 0.9% | | | |
| 40/75/12 wk, | 97.3 mg | 0.464 mg | 0.00477 | 92.80% | Imp. B: 0.18% | N/D |
| n = 3 | RSD: 1.2% | RSD: 2.0% | RSD: 1.1% | | | |
| 40/75/4 mo, | 98.1 mg | 0.482 mg | 0.00491 | 96.40% | Imp. B: 0.54% | N/D |
| n = 3 | RSD: 1.7% | RSD: 1.6% | RSD: 0.6% | | | |

TABLE 3
Stability of Meloxicam 0.47% with 0.5% PVA and 15% EtOH

| Stability | Spray | Spray | SC/SW | % Label | Impurity | Other |
|---|---|---|---|---|---|---|
| Condition | Weight | Content | Ratio | Claim | B & C | Impurity |
| Initial | N/A | Bulk 0.470% | N/A | 94.00% | N/D | Unknown: |
| | | RSD: 0.4% | | | | 0.5% |
| 25/60/2 wk, | 98.5 mg | 0.474 mg | 0.00481 | 94.70% | <0.10% | N/D |
| n = 3 | RSD: 1.4% | RSD: 1.5% | RSD: 0.2% | | | |
| 25/60/4 wk, | 98.5 mg | 0.485 mg | 0.00492 | 97.00% | <0.10% | N/D |
| n = 3 | RSD: 1.2% | RSD: 1.5% | RSD: 0.3% | | | |
| 25/60/8 wk, | 93.7 mg | 0.454 mg | 0.00485 | 90.90% | Imp. B: 0.14% | N/D |
| n = 3 | RSD: 8.4% | RSD: 7.7% | RSD: 0.7% | | | |
| 25/60/12 wk, | 98.5 mg | 0.481 mg | 0.00488 | 96.20% | <0.10% | N/D |
| n = 3 | RSD: 1.9% | RSD: 1.1% | RSD: 0.8% | | | |
| 25/60/4 mo, | 98.8 mg | 0.493 mg | 0.00499 | 98.50% | <0.10% | N/D |
| n = 3 | RSD: 1.2% | RSD: 2.6% | RSD: 3.8% | | | |
| 25/60/4 mo, cycle | 98.1 mg | 0.467 mg | 0.00476 | 93.40% | <0.10% | N/D |
| n = 3 | RSD: 1.9% | RSD: 1.9% | RSD: 1.0% | | | |
| 40/75/2 wk, | 95.2 mg | 0.459 mg | 0.00482 | 91.80% | <0.10% | N/D |
| n = 3 | RSD: 4.6% | RSD: 4.4% | RSD: 0.2% | | | |
| 40/75/4 wk, | 98.5 mg | 0.490 mg | 0.00497 | 97.90% | <0.10% | N/D |
| n = 3 | RSD: 1.1% | RSD: 1.2% | RSD: 0.2% | | | |
| 40/75/8 wk, | 96.6 mg | 0.468 mg | 0.00484 | 93.70% | <0.10% | N/D |
| n = 3 | RSD: 2.7% | RSD: 3.3% | RSD: 1.4% | | | |
| 40/75/12 wk, | 97.4 mg | 0.469 mg | 0.00482 | 93.80% | Imp. B: 0.12% | N/D |
| n = 3 | RSD: 3.0% | RSD: 3.2% | RSD: 0.9% | | | |
| 40/75/4 mo, | 100.5 mg | 0.504 mg | 0.00501 | 100.80% | Imp. B: 0.35% | N/D |
| n = 3 | RSD: 0.6% | RSD: 2.9% | RSD: 2.9% | | | |

TABLE 4
Stability of Meloxicam 0.47% with 0.5% PVA and 15% EtOH in Borate Buffer

| | | 4 week | 2 month | 2 week | 4 week 4 | 2 month |
|---|---|---|---|---|---|---|
| | | 25° C./ | 25° C./ | 40° C./ | 0° C./ | 40° C./ |
| Test | T0 | 60% RH | 60% RH | 75% RH | 75% RH | 75% RH |
| Assay of | 0.473% (w/w) | 0.467% (w/w) | 0.473% (w/w) | 0.473% (w/w) | 0.469% (w/w) | 0.472% (w/w) |
| Formulation | 100.5% LC | 99.3% LC | 100.5% LC | 100.7% LC | 99.8% LC | 100.4% LC |
| Conc. (n = 3) | % RSD: 0.05 | % RSD: 0.77 | % RSD: 0.45 | % RSD: 0.17 | % RSD: 0.03 | % RSD: 0.13 |
| Impurity/ | Unknown: | Unknown: | Unknown: | Unknown: | Unknown: | Unknown: |
| Degradent | 0.11% | 0.09% | 0.06% | 0.11% | 0.08% | 0.10% |
| | | | Imp. A: 0.04% | | | Imp. A: 0.05% |
| pH | 7.94 | N/A | 7.79 | N/A | N/A | 7.25 |
| Spray Weight | 97.0 mg | 97.5 mg | N/A | 97.2 mg | 96.8 mg | N/A |
| Uniformity | % RSD: 0.94 | % RSD: 0.77 | | % RSD: 2.81 | % RSD: 3.58 | |
| (n = 10) | | | | | | |
| Spray Content | 0.459 mg | 0.463 mg | N/A | 0.466 mg | 0.457 mg | N/A |
| Uniformity | 97.6% LC | 98.5% LC | | 99.3% LC% | 97.3% LC | |
| (n = 10) | % RSD: 0.85 | % RSD: 0.54 | | % RSD: 2.71 | % RSD: 3.36 | |
| Impurity/ | Unknown: | Unknown: | N/A | Unknown: | Unknown: | N/A |
| Degradent | 0.12% | 0.09% | | 0.11% | 0.10% | |
| Droplet Size | | | | | | |
| Distribution: | | | | | | |
| %, <10 μm: | 0.97% | 0.83% | N/A | 0.71% | 0.72% | N/A |
| D10: | 32.07 | 34.98 | N/A | 51.75 | 45.18 | N/A |
| D50: | 88.79 | 100.45 | N/A | 130.70 | 122.32 | N/A |
| D90: | 141.02 | 150.59 | N/A | 182.63 | 171.31 | N/A |
| Spam: | 1.23 | 1.15 | N/A | 1.00 | 1.03 | N/A |

TABLE 5
Spray Characterization Study of Meloxicam 0.47% with 0.5% PVA and 15% EtOH

| Sample ID | <10 μmm | Dv(10) | Dv(50) | Dv(90) | Span | Spray Angle | Ovality |
|---|---|---|---|---|---|---|---|
| 11 | 1.59% | 23.09 | 45.53 | 102.73 | 1.75 | 27.9 | 1.343 |
| 12 | 1.59% | 22.96 | 46.50 | 106.95 | 1.81 | 39.0 | 1.154 |
| 21 | 1.64% | 23.18 | 49.14 | 108.73 | 1.74 | 42.5 | 1.211 |
| 22 | 1.69% | 22.86 | 48.16 | 108.63 | 1.78 | 29.9 | 1.205 |
| 31 | 1.52% | 23.45 | 47.92 | 104.45 | 1.69 | 31.2 | 1.186 |
| 32 | 1.57% | 23.02 | 48.02 | 103.92 | 1.68 | 40.5 | 1.468 |
| AVERAGE | 1.60% | 23.09 | 47.55 | 105.90 | 1.74 | 35.17 | 1.26 |
| STD. DEV | 0.06% | 0.21 | 1.30 | 2.55 | 0.05 | 6.22 | 0.12 |
| RSD | 3.7 | 0.9 | 2.7 | 2.4 | 2.9 | 17.7 | 9.5 |

TABLE 6
Spray Characterization Study of Meloxicam 0.47% with 0.25% PVA and 15% EtOH

| Sample ID | <10 μmm | Dv(10) | Dv(50) | Dv(90) | Span | Spray Angle | Ovality |
|---|---|---|---|---|---|---|---|
| 11 | 2.62% | 18.70 | 39.08 | 100.01 | 2.08 | 29.1 | 1.298 |
| 12 | 2.46% | 19.38 | 39.41 | 96.70 | 1.96 | 29.6 | 1.426 |
| 21 | 2.36% | 19.36 | 40.27 | 102.34 | 2.06 | 37.9 | 1.336 |
| 22 | 2.33% | 19.58 | 39.98 | 96.13 | 1.91 | 44.0 | 1.818 |
| 31 | 2.34% | 19.62 | 41.67 | 99.86 | 1.93 | 29.2 | 1.593 |
| 32 | 2.41% | 19.22 | 40.80 | 98.49 | 1.94 | 31.2 | 1.611 |
| AVERAGE | 2.42% | 19.31 | 40.20 | 98.92 | 1.98 | 33.50 | 1.51 |
| STD. DEV | 0.11% | 0.33 | 0.94 | 2.31 | 0.07 | 6.13 | 0.20 |
| RSD | 4.5 | 1.7 | 2.3 | 2.3 | 3.6 | 18.3 | 13.0 |

The above description and examples are only illustrative of preferred embodiments which achieve one or more objects, features, and advantages of the present invention, and it is not intended that the present invention be limited thereto. Any modifications of the present invention which come within the spirit and scope of the following claims is considered part of the present invention.

1-25. (canceled)

26. A method of treating a condition in a human or non-human animal, comprising spraying a unit dose volume of about 25 to 400 mcl of a pharmaceutical composition on the oral mucosa of the animal, wherein the spray has a median particle size diameter of about 40 to about 100 microns, the composition comprises an active pharmaceutical agent, a solvent, and a viscosity modifying agent, and the active agent is absorbed through the oral mucosa to provide a therapeutically effective amount of the active agent to the systemic circulatory system to alleviate said condition.

27. The method of claim 26, wherein the composition comprises meloxicam, an alcohol, and a buffer.

28. The method of claim 26, wherein the active pharmaceutical agent is selected from the group consisting of meloxicam, celecoxib, ondansetron, sumatriptan, zolpidem, tizanidine, ropinerole, insulin, glucose, and nitroglycerine

29. The method of claim 27, wherein the amount of meloxicam is from about 0.1 to about 2% w/w.

30. The method of claim 29, wherein the amount of meloxicam is from about 0.25 to about 1% w/w.

31. The method of claim 30, wherein the amount of meloxicam is about 0.47% w/w.

32. The method of claim 26, wherein the spray volume is about 50 to 400 microliters.

33. The method of claim 32, wherein the spray volume is about 50 to 200 microliters.

34. The method of claim 33, wherein the spray volume is about 100 microliters.

35. The method of claim 33, wherein the spray volume is about 50 microliters.

36. The method of claim 32, wherein the spray volume is about 200 microliters.

37. The method of claim 26, wherein the meloxicam is administered in a dose from about 0.025 milligrams to about 2 milligrams per kilogram per day.

38. The method of claim 37, wherein the meloxicam is administered in a dose from about 0.05 milligrams to about 1 milligrams per kilogram per day.

39. The method of claim 38, wherein the meloxicam is administered in a dose from about 0.1 milligrams to about 0.2 milligrams per kilogram per day.

40. The method of claim 27, wherein the condition is selected from the group consisting of osteoarthritis, rheumatoid arthritis, inflammation, gout, and pain, and the composition comprises about 0.1 to 2% w/w of meloxicam, about 1 to 10 mg/g polyvinyl alcohol, and 100-300 mg/g of ethyl alcohol.
